(19) United States
(12) Patent Application Publication
(10) Pub. No.: US 2005/0203416 A1
(43) Pub. Date: Sep. 15, 2005

(54) Extended, ultrasound real time 2D imaging probe for insertion into the body

(76) Inventors: Bjorn A.J. Angelsen, Trondheim (NO); Tonni F. Johansen, Trondheim (NO)

Correspondence Address: Lance J. Lieberman, Esq.;Cohen, Pontani, Lieberman & Pavane, Suite 1210, 551 Fifth Avenue, New York, NY, 10176, US

(21) Appl. No.: 11/077,521

(22) Filed: Mar. 10, 2005

Related U.S. Application Data
(60) Provisional application No. 60/551,736, filed on Mar. 10, 2004.

(51) Int. Cl. A61B 8/14

(52) U.S. Cl. 600/463

(57) ABSTRACT

An ultrasound probe with a distal probe tip that can be inserted into the body for real time 2D ultrasound imaging from said probe tip, where said 2D image can be both in the forwards direction from the probe tip and at an angle to the probe tip. The ultrasound beam is generated with one of a single element transducer, and an annular array transducer, and scanned laterally through mechanically movement of the array. The mechanical movement is either achieved by rotation of the array via a flexible wire, or through wobbling of the array, for example through hydraulic actuation. The probe can be made flexible or stiff, where the flexible embodiment is particularly interesting for catheter imaging in the heart and vessels, and the stiff embodiment has applications in minimal invasive surgery and other procedures. The probe design allows for low cost manufacturing which allows factory sterilized probes to be disposed after use.

### RELATED APPLICATIONS

This application claims priority from U.S. Provisional Patent Application Ser. No. 60/551,736 which was filed on Mar. 10, 2004.

### BACKGROUND OF THE INVENTION

1. Field of the Invention

The invention relates to methods, ultrasound probes, and instrumentation for real time 2D imaging from the tip of an ultrasound probe that can be inserted into the body, either through natural openings or through surgical wounds.

2. Description of the Related Art

Real time (Rt) two-dimensional (2D) ultrasound imaging around the tip of an ultrasound probe that can be inserted into the body, is in many situations a sought after tool, both for diagnosis and for guidance of procedures. Examples of such procedures are placement of devices in vessels, heart ventricles and atria, guidance of electrophysiology ablation, or guidance in minimal invasive surgery. In these cases, the ultrasound probe gets in direct contact with the blood path, and it is then a great advantage to use factory-sterilized, disposable probes. This requires that the manufacturing cost of the probes can be kept low.

There is further a need for the probe to be flexible, for example for insertion into the vessels and the heart as a catheter. In this situation one could also want to control flexing of the tip from the external instrument. In other situations, like endoscopic surgery, one would like to have a stiff probe. A limited diameter puts a limit to the number of signal wires that can run along the probe.

### SUMMARY OF THE INVENTION

The present invention provides a solution to these problems by using mechanical scanning of the ultrasound beam from a single element transducer with fixed focus, or an annular transducer array with depth steered focus. For the annular array, one can conveniently use solutions as described in U.S. Pat. No. 6,540,677, to increase the sensitivity and reduce the number of wires connecting between the probe tip and the external imaging instrument. Two embodiments for mechanical scanning of the probe is proposed:

1. In the first embodiment, the transducer array is mounted at the tip of a rotating wire, and the beam is pointed at an acute angle to the rotation axis so that the beam is scanned along a conic surface in the forwards direction from the probe tip. The conic image is then divided into sub sectors and visualized as several plane sectors on the image screen. In a variation of embodiment a second transducer is mounted at close to right angle to the rotating probe tip, for additional imaging at a close to cross sectional plane of the probe.

2. In a second embodiment, the transducer array is mounted at a wobbling structure at the tip of the array, so that the ultrasound beam is scanned within a plane 2D sector. The wobbling is conveniently driven by hydraulic means. The 2D scan plane can be directed both in the forwards direction from the probe tip and at an angle to the probe tip.

Sensors to measure the angular position of the array, both in relation to the probe tip, and in relation to the external world, can be mounted at the array to be used in a feedback loop to control the scanning speed of the beam, and/or to trigger the image beams so that they are spread over the image with adequate angular distance, or the angle is used in the reconstruction of the image if the angular distance between the image beams varies over the image.

For limited movement velocity of the imaging object, one can obtain dynamic focusing of the ultrasound beam in the 2D azimuth scan plane by linear combination of the received RF signal from neighboring receive beams. Dynamic focusing in the elevation direction is best done with annular arrays, which then also would give dynamic focusing in the azimuth plane also.

The probes can be made both flexible and stiff, for best adaption to the application. The tip of the flexible probe can be direction steered (flexed) through wires along the periphery of the probe that are stretched/released through handles at the outside instrument.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings:

FIG. 1 shows an overview of a real time 2D imaging system with an probe according to the invention, and

FIG. 2 shows an example embodiment of the distal tip of a flexible, probe according to the invention where the ultrasound beam is scanned within a forward cone, and

FIG. 3 shows an example 2D display of the conic image on a screen, and

FIG. 4 shows yet another arrangement with two rotating transducers according to the invention, and

FIG. 5 shows yet another method of 2D scanning of the ultrasound beam within a plane 2D sector from the distal tip of the probe, according to the invention, and

FIG. 6 shows an example of an optical angular position resolver for measuring the mechanical rotation of the array in a probe tip like displayed in FIGS. 2 and 4, and

FIG. 7 shows an example of an optical angular position resolver for measuring the angular wobbling of the array for a probe tip of the type shown in FIG. 5.

### DETAILED DESCRIPTION OF THE PRESENTLY PREFERRED EMBODIMENTS

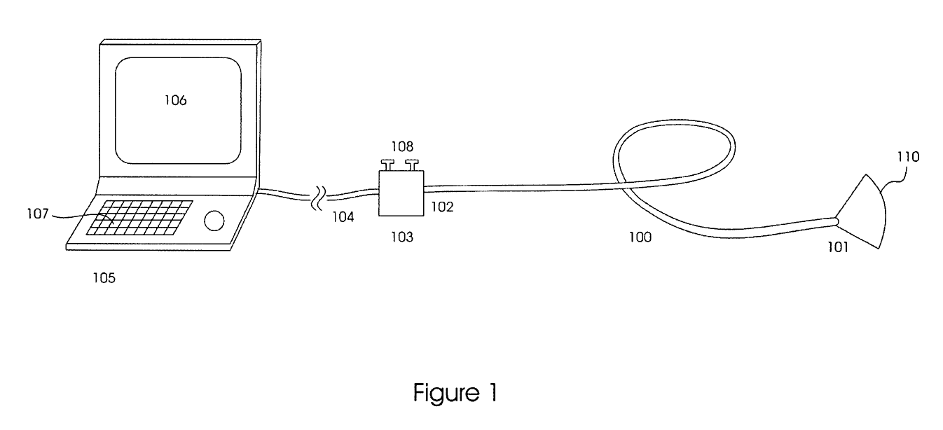

The invention relates to an ultrasound real time 2D imaging system, which in a typical embodiment is composed of the components shown in FIG. 1, where 100 shows an elongated imaging probe with a distal imaging tip 101 and a proximal end 102 that is connected to an utility console interface 103. The imaging ultrasound beam is transmitted from the distal tip of the probe enabled to be scanned within a 2D region to be imaged, for example illustrated as 110. The invention specially relates to methods of scanning the ultrasound beam within the 2D region 110, from the distal tip of such an elongated probe. The utility interface further connects via the cable 104, the probe signals to an ultrasound imaging instrument 105. The imaging instrument has an image display screen 106 for visualization of the images and also other information, and a key board interface 107 for user control of the instrument.

In this particular embodiment, the imaging probe 100 is a particularly flexible catheter probe for example allowing double curving of the probe, which has advantages for imaging inside tortuous vessels and the heart cavities. For these applications one would also want the region close to the tip to be more flexible than the proximal region of the probe, as the tortuous flexing is mainly necessary in the distal region, while less flexibility of the proximal region helps in manipulation of the probe. In other applications, the probe can be much less flexible, close to stiff, for example in minimally invasive surgery where the probes would be inserted through a trocar. For the flexible probe, one can in some embodiments stretch wires along the periphery of the probe, where the wires can systematically be stretched and released by control organs 108 at the utility interface 103 for flexing the tip of the probe in one or two directions.

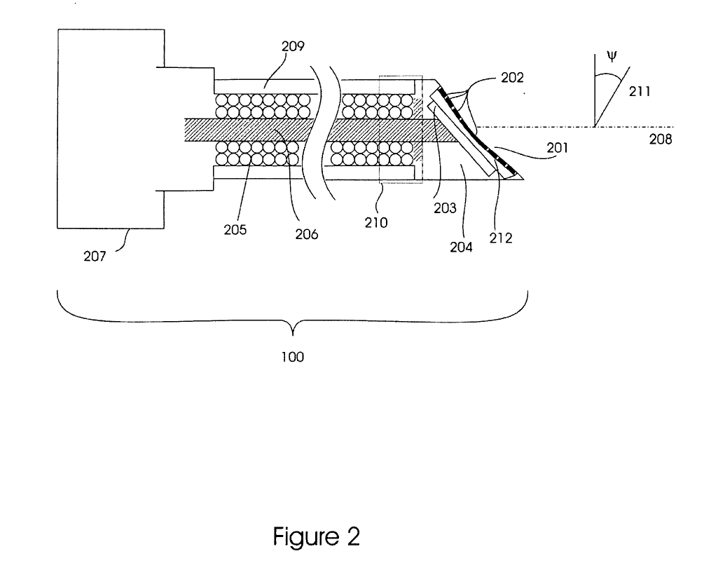

FIG. 2 shows a first example embodiment according to the invention of the distal tip 101 of such an elongated probe 100. In this Figure, 201 shows an annular array transducer with array elements 202. The array elements 202 are in this embodiment electrically connected to an electronic circuit 203, with an acoustically isolating material (backing material) 212 between the array and the circuit, to avoid ringing acoustic pulses from the back side of the array. The circuit 203 typically contains receiver amplifiers with switching circuits between transmit and receive of the ultrasound pulses. In some embodiments it can also contain steerable or selectable delay elements to combine the signals from neighboring elements into a transmit beam with selectable focus and/or a dynamically focused receive beam. The delay elements can both be electronic or implemented acoustically with delay material in front of each array element. The electronically steered focusing can both be done in the circuit 203 at the tip of the probe, for example according to the methods described in U.S. Pat. No. 6,540,677, or the focusing can be done at the external instrument which requires that all the element signals are connected from the array to the external instrument with cable wires throughout the whole length of the probe.

When the imaging object has limited movement velocity, the number of wires between the imaging tip and the external imaging instrument can also be reduced with synthetic focusing techniques, for example where one in a sequence image with the different elements in the probe, and combine these signals into a beam that is focused at all depths with focus width determined by the active aperture of the array. Synthetic focusing in the azimuth direction can also be obtained by linear combination (filtering) of the RF-signal of neighboring, fixed focus or unfocused azimuth beams.

The array 201 and the circuit 203 are mounted in an array holder unit 204 that is connected to a flexible rotation cable 205 typically made of double helix spun wires, like a speedometer wire. The rotation cable 205 has a core of electric cable wires 206 that connects the array and circuit to the external utility console 103, as shown in FIG. 1. The wire is on the distal end connected to a motor 207 in the utility console, and transmits the motor rotation to rotation of the transducer array 201 around the cable axis 208. The rotating cable would typically be covered with a plastic sheath 209, but this sheath could in some embodiments be left out. One should note that in some embodiments, the electronic circuit 203 can also be left out, and the cable wires 206 would then connect directly to the array elements 202.

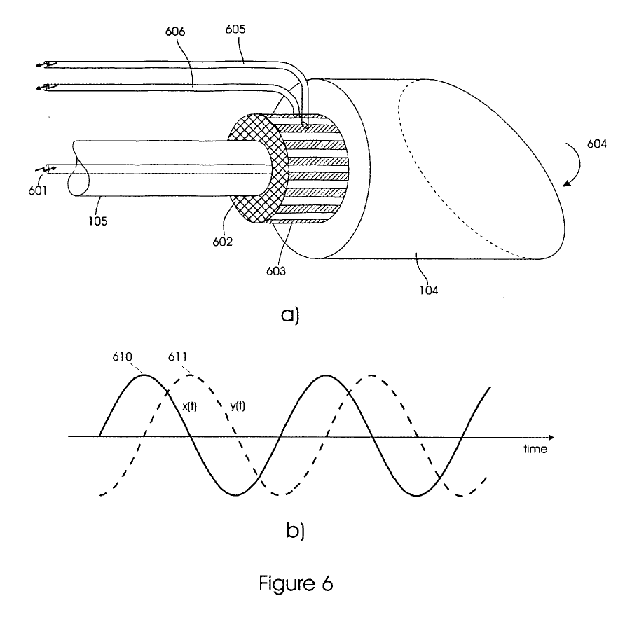

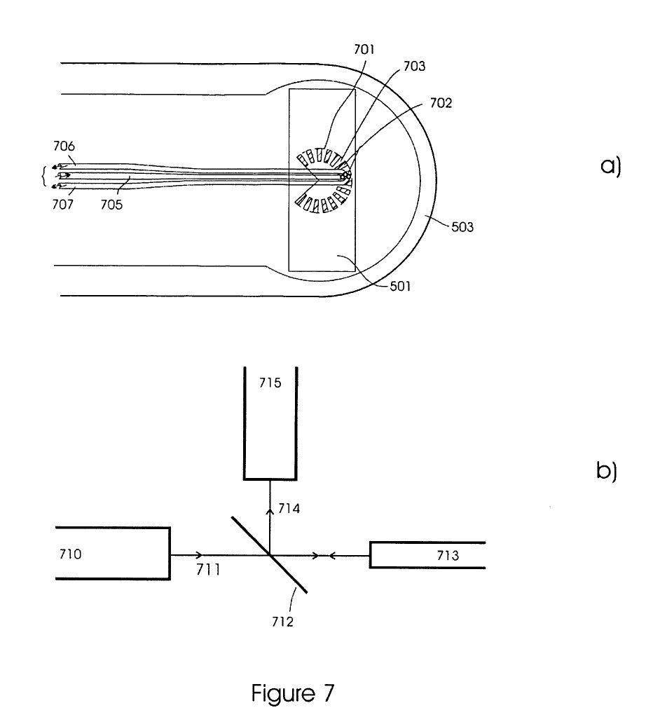

For accurate sensing of the angular direction of the array, a position sensor 210 would typically be mounted at the probe tip to measure the rotation ψ, indicated as 211, of the array holder 204 and array 201 in relation to the catheter sheath 209 . This position sensor could typically be of optical types like described in FIGS. 6 and 7. Other methods, like electromagnetic angular position sensors could also be used, or one could even use electromagnetic coupling between a sensor at the array holder unit and one sensor in a more fixed location in the patient body, or external to the external to the patient body, both for measuring the angular direction of the ultrasound beam, but also for measuring the x,y,z position of the ultrasound array. Accurate monitoring of the angular direction of the array and beam can be used to trigger that transmit for the image beams at selected angles, but also in a feed back system to obtain close to constant rotation speed of the transducer array. If the angular direction of the different image beams is irregularly spaced, the measured angular position of each image beam can be used in the image reconstruction to avoid image deformation due to this irregularity of the beam positions.

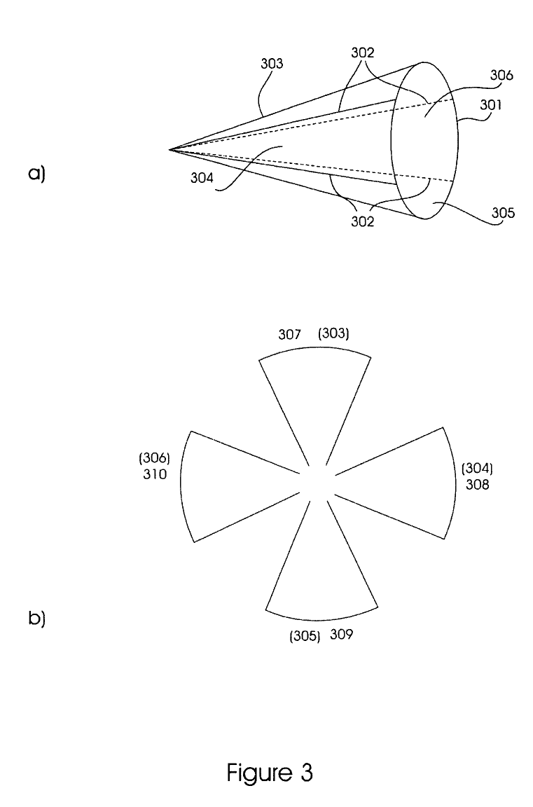

An example of visualization of the 2D conic image data on a flat screen, is shown in FIG. 3. 301 shows the conic surface across which the beam is scanned, where the 2D image can be visualised. This surface can further be divided for example along 4 radial lines 302 to be separated into 4 surface regions 303, 304, 305, and 306. These surface regions are then projected onto the plane sectors 307, 308, 309, and 310 displayed on the image screen in the same sequence. The images are typically shown as grey scale images for the amplitude of the reflections that gives a tissue image, or in a color scale for movement velocities of the object, according to well-known principles.

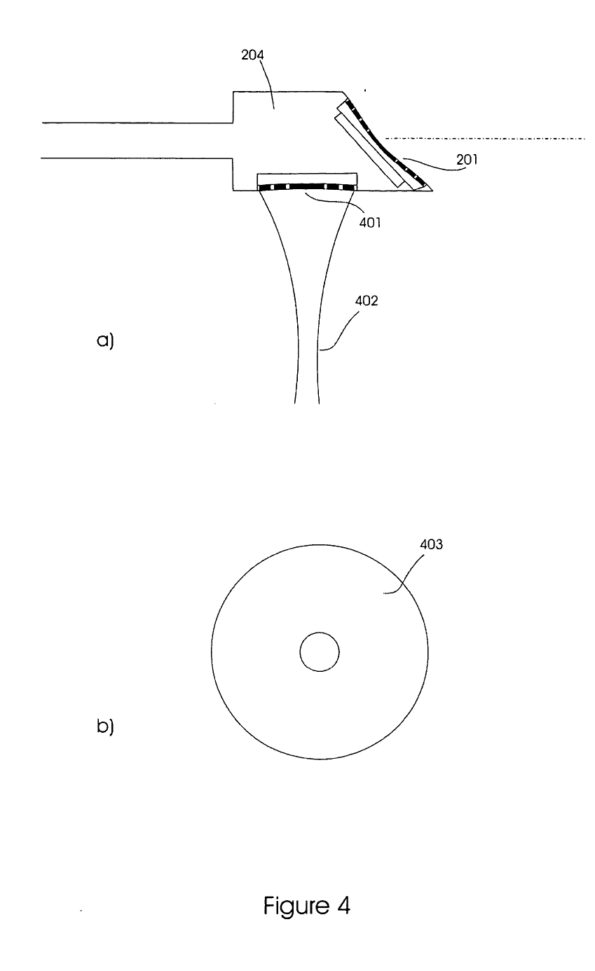

For various applications, for example for measurement of a vessel cross section or observations of the cardiac valves, it is advantageous in addition to the forward cone to show a cross sectional image around the probe tip. This can be achieved as shown in FIG. 4, which shows a similar probe tip as in FIG. 2, but with an added transducer array 401 with a beam 402 at the circumference of the rotating array holder unit 204. This transducer can again be a single element transducer or an annular transducer array, similar to the forward looking array 201. The 2D image would then be displayed as 403 on the screen, typically together with the forward looking image as in FIG. 3. Due to the angular difference between the forward and transverse looking beams from the arrays 201 and 401, one could transmit the image pulses for these arrays at the same time, and record the back scattered signals in parallel. However, this will generate some acoustic cross talk noise between the two beams, and also requires parallel electronics to operate the arrays. Allowing for some reduction in image frame rate, one would rather operate the two arrays with interleaved time multiplexing, transmitting each second pulse on array 201 and the other pulses on array 401.

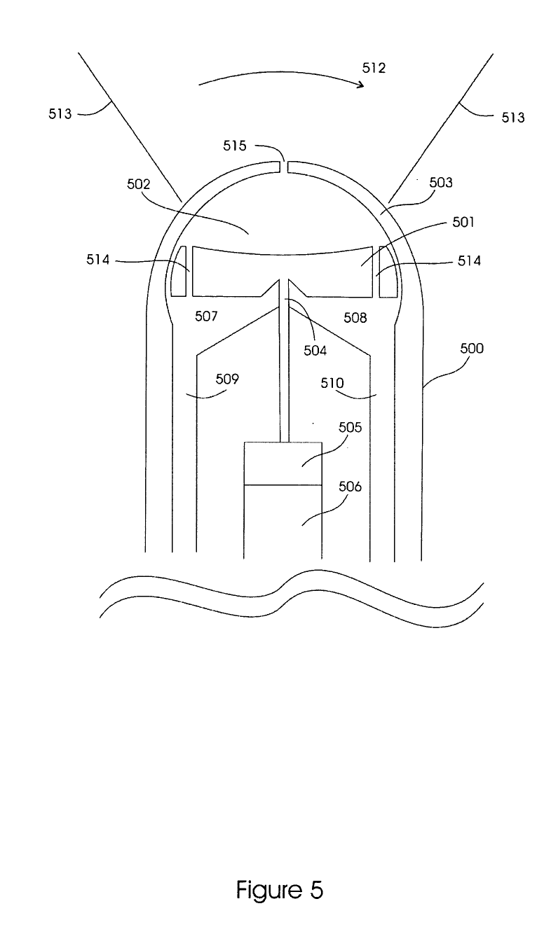

Another embodiment for 2D scanning of the ultrasound beam according to the invention, is shown in FIG. 5, where 501 shows the array holder, possibly including the integrated circuit 203, that is enclosed in a sub-spherical dome 503. The assembly 501 is connected to a flexible member 504 that locates the assembly in the middle of the dome and also feeds electric signal wires from the array and electronic circuit to the imaging instrument. The member 504 can for example be made as a printed flex circuit or similar structure. The signal wires can connect to a more convenient type of cable 506 at the interface 505 to be fed throughout the probe to connect to the utility console 103 of FIG. 1.

The probe contains in this example embodiment two hydraulic channels 509 and 510 that can inject or remove fluid from the chambers 507 and 508, that are separated by the flexing member 504. In normal scanning operation, the interior compartments 502, 507, and 508 are filled with a fluid, preferable water with physiological composition. Injecting fluid through the tube 509 into compartment 507 while removing similar amounts of fluid through tube 510 from compartment 508 causes the array/circuit assembly 501 to rotate in the clockwise direction indicated by the arrow 512. The opposite rotation is obtained by injecting fluid through tube 510 into chamber 508 while removing a similar amount of fluid through tube 509 from chamber 507.

For simplified filling of the chambers 502, 507, and 508 with fluid, without introducing air bubbles, a continuous forward filling with fluid is obtained by the channels 514 that feeds fluid from the compartments 507 and 508 into the compartment 502, while the channel 515 feeds fluid from the compartment 502 to the outside front of the probe dome. This continuous flow of fluid to the front of the dome, improves acoustic contact between the dome and the object contact surface, or can spill into the blood when the probe is inserted into a blood-filled region. In other embodiments, the draining of the fluid from compartment 502 can in addition or instead be done through the probe to its proximal, outside end, by an additional specific channel through the probe from the distal to the proximal end.

The probe is on its proximal end connected electrically and hydraulically to the utility console 103 of FIG. 1, which for this embodiment also contains a hydraulic pumping and control system that injects or removes fluid through the channels 509 and 510 and provides the wobbling motion of the array assembly 501. The array can typically be an annular array or a single element transducer with a fixed focus. This provides a two-dimensional scanning of the ultrasound beam in the forwards direction from the probe tip, illustrated as the sector 513. It is clear though that the hydraulic method of beam scanning described in this Figure also nicely allows angling of the 2D scan in relation to the probe tip axis. With these scan methods, the 2D image based on the back scattered signals can be visualized as a standard 2D sector grey scale image of the tissue scattering and/or a color 2D sector image of object velocities.

To avoid geometric distortions of the image in the direction of the mechanical scan, one can conveniently use an angular position sensor of the moving array/circuit assembly at the tip of the probe. Such position sensors can be based on optical or electromagnetic principles according to known methods, and for sake of example FIG. 6 illustrates an optical position sensor for the rotating scan system of FIGS. 2 and 4, and FIG. 7 illustrate an optical position sensor for the wobbling scan system in FIG. 5.

FIG. 6a shows the rotating array holder 104 with the rotating drive cable 105, that rotates the array in the direction indicated by 604. The rotating drive cable contains in this example embodiment also an optical fiber 601 that feeds light into a transparent sub-part 602 of the array holder. The surface of the sub-part 602 is partly covered with a light inhibiting film at the end face and also at grating lines 603 in a periodic pattern along the circumference of 602 that inhibits light to shine out through the circumference, while between the grating lines the light is allowed to shine through. The distance between the grating lines is equal to the width of the grating lines within the accuracy of the manufacturing.

Two optical fibers 605 and 606 picks up light that shines through the circumference of 602 and feeds the light back to the instrument where it is converted to electrical analog signals by for example photo transistors and subsequently converted to digital form for processing to accurately detect the rotational angle of the array holder 104. Example signals after the phototransistors for the two fibers are shown in FIG. 6b where 610 shows a typical signal x(t) from fiber 605, and 611 shows a typical signal y(t) from fiber 606. Due to spread of the light, the signals are close to sinusoidal in shape. The two fibers 605 and 606 have a distance between each other close to ¼ of the period of the grating lines, which gives close to 90 deg phase lag of y(t) in relation to x(t). An accurate resolving of the rotational angle ψ, can then for example be found by the following relation
ψ(t)=F{x(t), y(t)} (1)
where for many applications F{ } can be approximated by the inverse tangent as
ψ(t)=F{x(t), y(t)}=tan⁻¹{y(t)/x(t)} (2)

A similar optical position sensor for the wobbling system in FIG. 5, is shown in FIG. 7a, where 501 shows the array holder within the dome 503. In this example embodiment, a variable reflectance grating 701 composed of stripes 702 with high reflectance periodically arranged with stripes 703 of low reflectance. A triple optical fiber system 704 containing one fiber 705 for shining light onto the reflectance grating, and two fibers 706 and 707 for transmitting the light reflected from the grating to the instrument. The reflected light is detected and digitized in the instrument as for the position sensor in FIG. 6a. The distance between the pickup areas of fiber 706 and 707 is ¼ of the grating period, so that the signals in the two fibers 706 and 707 produces signals x(t) and y(t) as in FIG. 6b, which is further processed to resolve the angular position of the array holder similar to Eqs.(6,7).

In FIG. 6a is shown a position sensor with a transmitting grating, while it is clear to any one skilled in the art that a reflecting grating could equally well be used similar to the sensor in FIG. 7a, for which sensor one could also use a transmitting grating.

With two fibers that collects light that is 90 deg out of phase with each other (quadrature phase) one is able to resolve the direction of rotation. If one knows the rotation direction, it would be sufficient to have a single fiber for the reflected light, however, the conversion from light intensity to angle would be simplified by the use of two light signals with quadrature phase relationship.

The same fiber can also be used for transmitted and reflected light using for example a transmitting mirror as shown in FIG. 7b. The light source 710 shines a light beam 711 through a transmitting mirror 712 so that the light enters the fiber 713. The light reflected at the distal end of the fiber will then come out of the tip and be reflected at the mirror 712 so that the reflected light is separated into the beam 714 that hits the detector 715 and is converted to an electrical signal and digitized.

Other methods of angular position sensing can be based on electromagnetic methods where many such methods are known.

Using wide band or multi-band transducers based on ceramic films, for example as described in U.S. Pat. No. 6,671,692, one can operate the ultrasound transducer both in a low frequency band for an overview image with large penetration, and in a high frequency band for a short range image with improved resolution. The overview image could for example be used to guide ones way in the cardiac chambers to move the probe tip close to an electrophysiology ablation scar, and then evaluate the scar with the high resolution short range image. Similarly could the long range image be used to get an overview of the movement of native heart valves to evaluate best procedure for valve repair or valve replacement, while the short range image can be used to evaluate details in valve morphology.

It is also expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment as a general matter of design choice. It is the intention, therefore, to be limited only as indicated by the scope of the claims appended hereto.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. An ultrasound imaging probe with a distal imaging tip to be inserted into a body, and a proximal end, opposite along the probe to said distal tip, to be connected to an external ultrasound imaging instrument outside said body, comprising
a rotating shaft that runs along the probe from its proximal to its distal end, the proximal end of said shaft being connected to a rotating motor, and
at the distal end of said rotating shaft there is mounted an ultrasound transducer or annular transducer array that transmits and receives ultrasound imaging beams, mounted so that said beams form an acute angle in the forwards direction to the rotating axis of said distal tip of the shaft,
so that
rotation of said shaft by said motor provides a sweeping of said ultrasound beam within a conic surface in the forwards direction from said distal probe tip for real time 2D ultrasound imaging along said conic surface.

2. An ultrasound imaging probe according to claim 1, where said shaft is a dual helix wire spun around an electrical cable that connects the signals from said transducer or transducer array to said external imaging instrument.

3. An ultrasound imaging probe according to claim 1, where the back scattered ultrasound signal is analyzed to form one or both of a grey scale tissue image, and a color Doppler image of moving scatterers in the region along the forward scanning cone, where for display of said images said scanning cone is divided into sector regions and each region is displayed as plane 2D sectors within a circular region so that the position relation between said cone sectors is maintained in said image.

4. An ultrasound imaging probe according to claim 1, where in addition to said ultrasound transducer or transducer array that is sweeping an ultrasound beam along said forward cone surface, a second ultrasound transducer or transducer array is mounted at said rotating shaft tip, so that said ultrasound transducer or transducer array radiates or receives ultrasound waves along imaging beams that have a larger angle to the rotation axis of said distal shaft tip than said first imaging beams, so that said second ultrasound transducer or transducer array can be used to obtain real time 2D ultrasound images along a surface with larger angle to the rotation axis of said distal shaft tip.

5. An ultrasound imaging probe with a distal imaging tip to be inserted into a body, and a proximal end, opposite along the probe to said distal tip, to be connected to an external ultrasound imaging instrument outside said body, comprising
an ultrasound transducer or transducer array enabled to both transmit and receive ultrasound waves along imaging beams, said ultrasound transducer or transducer array being mounted to a holder structure at said distal probe tip, where
said holder structure can be rotated back and forth in a wobbling manner by hydraulic means where hydraulic fluid is injected through at least one channel that rides along the probe from said proximal to said distal end, and
said proximal end of said channel are connected to a hydraulic pumping system that is enabled to pump hydraulic fluid through said at least one channel,
so that
back and forth wobbling of said holder and transducer array by said hydraulic system provides a sweeping of said imaging beam within a 2D sector from said probe tip, for real time 2D imaging within said sector.

6. An ultrasound imaging probe according to claim 5, where said 2D sector is directed in the forwards direction of said probe tip.

7. An ultrasound imaging probe according to claim 5, where said 2D sector is directed at an angle to said probe tip axis.

8. An ultrasound imaging probe according to claim 5, where the probe hydraulic fluid fills the space around the array in the probe tip to function as an acoustic transmission fluid, and the tip contains one or more draining channels of the hydraulic fluid so that a continuous flow of fluid around the array is obtained to remove possible gas bubbles in the fluid around the array.

9. An ultrasound imaging probe according to claim 8, where at least one draining channel leads said hydraulic fluid to the exterior of said distal probe tip.

10. An ultrasound imaging probe according to claim 1, where said array is an annular array.

11. An ultrasound imaging probe according to claim 1, where said array is operable in multiple frequency bands, so that imaging with pulses in a low frequency band is used for a longer range overview image, and imaging with pulses in a high frequency band is used for near range, high resolution imaging.

12. An ultrasound imaging probe according to claim 11, where said low frequency and said high frequency pulses are transmitted in one of at the same time where the receive signal is filtered in the low and the high frequency range, and said low and high frequency pulses are transmitted interleaved in a sequence, so that real time 2D images in the high frequency and the low frequency range are visualized simultaneously.

13. An ultrasound imaging probe according to claim 1, where said distal tip of the probe contains integrated circuits with receiver amplifiers for high sensitivity imaging.

14. An ultrasound imaging probe according to claim 1, where said distal tip of the probe contains integrated circuits with receiver amplifiers and electronic and/or acoustic delay elements so that beam forming with a dynamic receive focus is done at the tip of the probe, so that the number of wires connecting said probe tip and said external imaging instrument can be less than the number of elements in said array.

15. An ultrasound imaging probe according to claim 1, where an angular position resolver is placed at said distal imaging tip to measure the angular position of said ultrasound transducer or transducer array in relation to the probe tip.

16. An ultrasound probe according to claim 1, where the angular rotation and position of said ultrasound transducer or transducer array is measured by electromagnetic sensors mounted on the array holder in relation to electromagnetic sensors inside or outside of the patient.

17. An elongated ultrasound imaging probe according to claim 15, where the angular position of said transducer or transducer array as measured by said angular position resolver is used in a feed back system to control the rotation/wobbling of said transducer or transducer array for close to constant rotation speed.

18. An ultrasound imaging probe according to claim 1, where the probe is flexible.

19. A flexible, ultrasound imaging probe according to claim 18, where wires run along the probe from said proximal to said distal end, so that by selective pulling and releasing tension of said wires at the proximal end, one can steer direction flexing of said distal end of the probe.

20. An ultrasound imaging probe according to claim 5, where said array is an annular array.

21. An ultrasound imaging probe according to claim 5, where said array is operable in multiple frequency bands, so that imaging with pulses in a low frequency band is used for a longer range overview image, and imaging with pulses in a high frequency band is used for near range, high resolution imaging.

22. An ultrasound imaging probe according to claim 5, where said distal tip of the probe contains integrated circuits with receiver amplifiers for high sensitivity imaging.

23. An ultrasound imaging probe according to claim 5, where said distal tip of the probe contains integrated circuits with receiver amplifiers and electronic and/or acoustic delay elements so that beam forming with a dynamic receive focus is done at the tip of the probe, so that the number of wires connecting said probe tip and said external imaging instrument can be less than the number of elements in said array.

24. An ultrasound imaging probe according to claim 5, where an angular position resolver is placed at said distal imaging tip to measure the angular position of said ultrasound transducer or transducer array in relation to the probe tip.

25. An ultrasound probe according to claim 1, where the angular rotation and position of said ultrasound transducer or transducer array is measured by electromagnetic sensors mounted on the array holder in relation to electromagnetic sensors inside or outside of the patient.

26. An elongated ultrasound imaging probe according to claim 16, where the angular position of said transducer or transducer array as measured by said angular position resolver is used in a feed back system to control the rotation/wobbling of said transducer or transducer array for close to constant rotation speed.

27. An ultrasound imaging probe according to claim 5, where the probe is flexible
